(19) United States
(12) Patent Application Publication
(10) Pub. No.: US 2013/0266486 A1
(43) Pub. Date: Oct. 10, 2013

(54) AIR FRESHENER

(71) Applicant: AROMATE INDUSTRIES CO., LTD., New Taipei City (TW)

(72) Inventor: Henry Wu, New Taipei City (TW)

(73) Assignee: Aromate Industries Co., Ltd., New Taipei City (TW)

(21) Appl. No.: 13/855,437

(22) Filed: Apr. 2, 2013

Related U.S. Application Data
(60) Provisional application No. 61/620,015, filed on Apr. 4, 2012.
(60) Provisional application No. 61/619,702, filed on May 3, 2012.

(51) Int. Cl. A61L 9/12

(52) U.S. Cl. CPC A61L 9/12
USPC 422/123

(57) ABSTRACT

An air freshener of the invention comprises a head having a receiving portion and a socket portion, a clip, having a ball portion rotatably and detachably mounting in the socket portion of the head, to mount the air freshener on an air vent and a scent portion received in the receiving portion of the head for diffusing fragrance.

### BACKGROUND OF THE INVENTION

1. Field of the Invention

The invention relates to an air freshener, especially to an air freshener that is compact in structure and easy to manufacture.

2. Description of the Related Art

There are a variety of air fresheners that can be placed in front of or inserted into an automobile's cool air vent to improve a car's interior air quality. With reference to FIGS. 12a and 12b, a conventional air freshener for a vehicle comprises a head 50, a clip 52 coupled to the head 50 extending perpendicularly outward from the head 50, and sized and shaped to be inserted into a vehicle air vent and clipped to a vehicle air vent cover such that the head 50 is adjacent to the vehicle air vent, and including a pair of vent rods 52a, 52b sized and shaped to be inserted into the vehicle air vent and having a plurality of protrusions 53, a pair of coherent elastomeric polymer bodies 54a, 54b having a plurality of apertures 55, each of the bodies 54a, 54b coupled to a different one of the vent rods 52a, 52b via coupling of the plurality of apertures 55 with the plurality of protrusions 53, and insertable into the vehicle air vent with the vent rods 52a, 52b and a scent material, interspersed within the bodies 54a, 54b, capable of diffusing out of the bodies 54a, 54b to surrounding air. However, the conventional air freshener is complicated in structure and not easy to manufacture and package. In addition, the bodies with the scent material can only act as a scent disperser, but are not quite able to benefit the eye-appealing effect of the product.

### SUMMARY OF THE INVENTION

It is an object of this invention to provide an air freshener which is compact in structure and easy to manufacture.

In order to achieve the above objects, the air freshener according to an embodiment of the present invention comprises a head having a receiving portion recessed from the peripheral surface of the head and a socket portion on its rear end, a clip having a ball portion on one end which is rotatably and detachably received in the socket portion of the head and a scent portion fitted to and received in the receiving portion of the head.

The present invention further provides an air freshener comprising a head having a receiving portion recessed from the peripheral surface of the head, a clip extruding from a rear end of the head and a scent portion fitted to and received in the receiving portion of the head.

In an embodiment of the present invention, the socket portion has four confining walls respectively extending from the rear end of the head and disposed along the periphery of the socket portion so as to form a cross-like gap therebetween which is slightly narrower in its center than the diameter of the ball portion such that the ball portion is pivotably held in the gap when the ball portion is inserted in the gap of the socket portion. The heights of the four confining walls extending from the rear end of the head are limited and the inner surfaces of the four confining walls are curved so that the swivel of the clip about the socket portion will not be interfered.

In an embodiment of the present invention, the socket portion has a first wall and a second wall respectively and opposingly extending from the rear end of the head, the first and second walls defining a gap therebetween which is slightly narrower than the diameter of the ball portion such that the ball portion is inserted and pivotably held in the gap of the socket portion. The heights of the first and second walls extending from the rear end of the head are limited and the contours of the first and second walls are curved so that the swivel of the clip about the socket portion will not be interfered.

In an embodiment of the present invention, the ball portion forms at least one rib thereon for increasing the friction between the socket portion and the ball portion.

In an embodiment of the present invention, the scent portion has a hollow portion fitted to and received in the receiving portion of the head.

In an embodiment of the present invention, the scent portion and the head is formed in any of the following shapes: circle, rectangle, oval, rhombus, square, doll-like shape, heart-shaped and irregular shape.

In an embodiment of the present invention, the scent portion has a core-out region to reduce a shrinkage rate during the formation of the scent portion.

In an embodiment of the present invention, the scent portion is at least partially in the form of an elastic band such that the scent portion is at least partially held in the receiving portion by the elasticity of the scent portion.

In an embodiment of the present invention, the head has an exhibition portion on its front end, and the exhibition portion has a pattern or words thereon.

In an embodiment of the present invention, the head has an exhibition portion on its front end, and the exhibition portion receives a label. The label can be covered with a transparent resin. The label can be relief or intaglio printed with desired patterns.

### DESCRIPTION OF THE DRAWINGS

FIG. 1 illustrates an exploded view of an air freshener according to the first embodiment of the present invention;

FIG. 2 illustrates another exploded view of the air freshener according to the embodiment of the present invention shown in FIG. 1;

FIG. 3 illustrates a schematic view showing the air freshener after assembly according to the embodiment of the present invention shown in FIG. 1;

FIG. 4 illustrates the second embodiment of the present invention;

FIG. 5 illustrates an exploded view of the third embodiment of the present invention;

FIG. 6 illustrates a schematic view of the third embodiment of the present invention;

FIGS. 7-9 illustrates schematic views of the fourth embodiment of the present invention;

FIG. 10 illustrates an example of the package of the air freshener according to the present invention;

FIG. 11 illustrates another example of the package of the air freshener according to the present invention; and

FIGS. 12a and 12b illustrate a conventional air freshener.

### DETAILED DESCRIPTION OF THE INVENTION

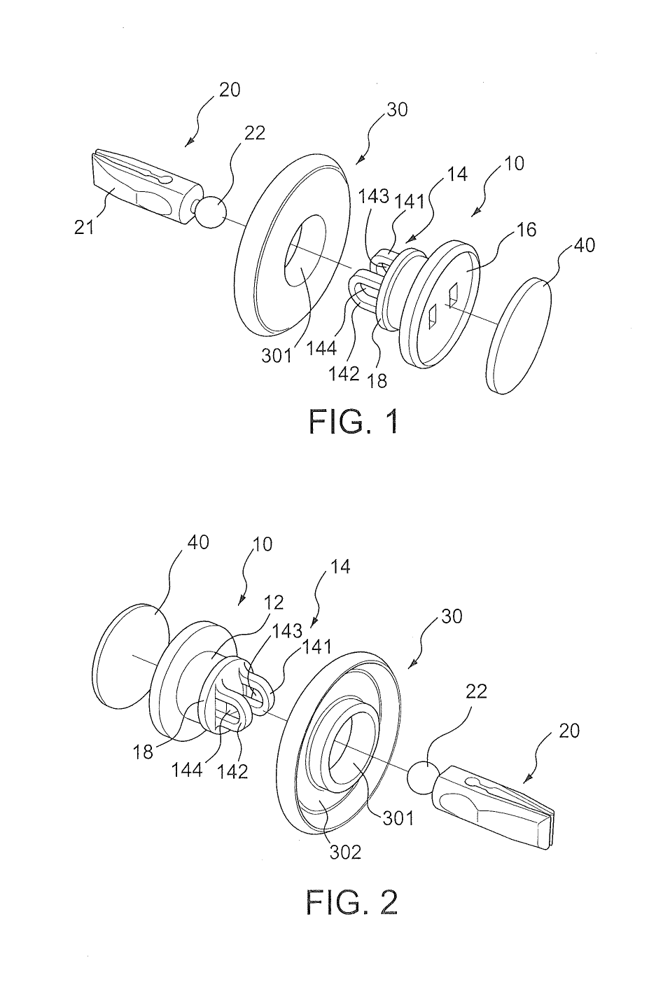

As shown in FIGS. 1 and 2, the air freshener of the first embodiment of the invention includes a head 10, a clip 20 and a scent portion 30. The head 10 has a front end which has an exhibition portion 16, a rear end which has a socket portion 14, and a receiving portion 12 which is recessed from the peripheral surface of the head 10 and between the front end and rear end for receiving the scent portion 30. The head 10 can further have a stopper 18. Said stopper 18 can be one or several protrusion formed between the receiving portion 12 and the rear end. The clip 20 is formed with two slender legs 21 with a zigzag shape and a slit between the legs so that the clip 20 is resilient and able to provide a clamping or retention force when it is inserted into the louver or fin of an air vent. The clip 20 further has a ball portion 22 on the end opposite to the legs 21.

The resilient clip 20 engages the head 10 by rotatably and detachably mounting the ball portion 22 of the clip 20 to the socket portion 14 of the head 10. The socket portion 14 is formed with a first wall 141 and a second wall 142 extending from the rear end of the head 10. The gap formed between the first wall 141 and the second wall 142 is slightly narrower than the diameter of the ball portion 22. As shown in FIGS. 1, 2 and 3, holes 143 and 144 are recessed from the inner surfaces of the first wall 141 and the second wall 142 respectively, such that the ball portion 22 can be pivotably held in the socket portion 14 by means of the gap formed between the first wall 141 and the second wall 142 and the holes 143 and 144 when the clip 20 is engaged with the head 10. The holes 143 and 144 can be through holes, as shown in FIGS. 1, 2 and 3. The first wall 141 and the second wall 142 and the connection portion between the ball portion 22 and the legs 21 of the clip 20 are so configured that the clip 20 can be pivoted from at least −90 to +90 degree angle (i.e., more than 180 degree pivot angle) along the inner surfaces of the first wall 141 and the second wall 142, and is pivotable along any other direction at a smaller angle. Take the first embodiment shown in FIGS. 1-3 as an example, the heights of the first wall 141 and the second wall 142 extending from the rear end of the head 10 are limited and the contours of the first and second walls 141, 142 are curved so that the swivel of the clip 20 about the socket portion 14 on the rear side of the head 10 will not be interfered.

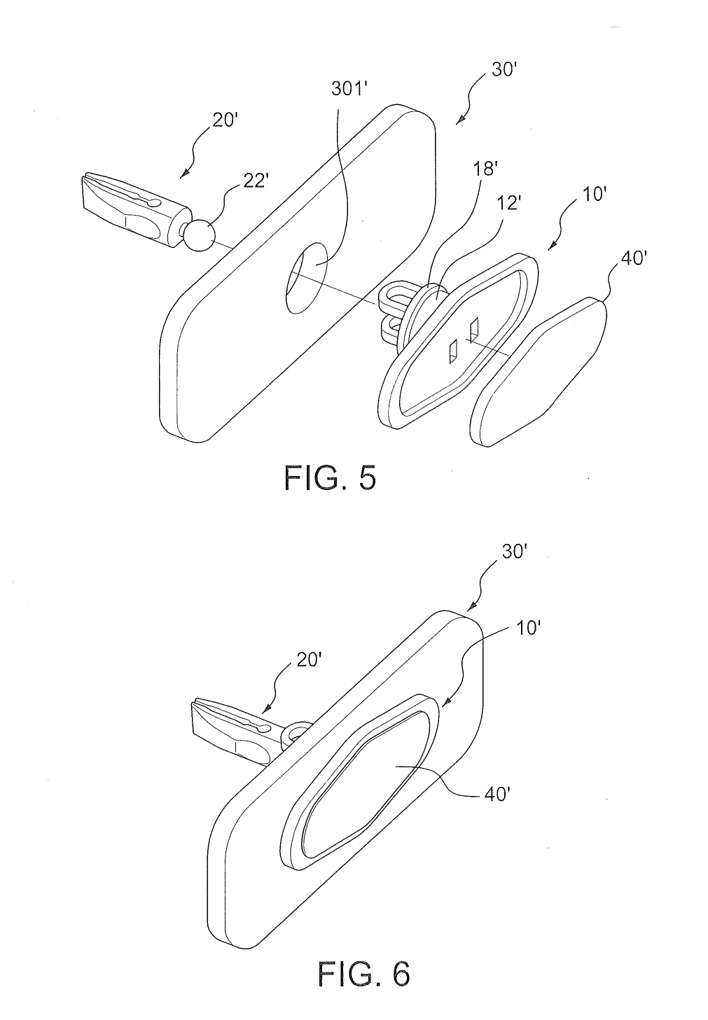

The air freshener of the invention can be inserted into and fixed to a louver or fin of an article, such as an air vent of an air conditioner, by the clip 20. Further, the front end of the head 10 can be of any shape to meet the user's demand. The scent portion 30 is made of polymer with any desired color for decoration; the scent portion 30 contains scent material for providing fragrance. The scent material is perfume oil or impregnated with perfume oil. The scent portion 30 can be formed in any of the shapes to meet the user's demand, such as circle, oval, rectangle, rhombus, square, doll-like shape, heart-shaped and irregular shape. The scent material may disperse or diffuse out of the material of the scent portion 30 into the air or atmosphere to be discernable or smelled. In FIGS. 1-3, the scent portion 30 is in the form of a circular band or a disk-like shape and can be at least slightly stretched because of the elasticity of the polymer material. The band-like or disk-like scent portion 30 has a hollow portion 301; the shape of the hollow portion 301 fits the receiving portion 12 of the head 10. In assembling the head 10 and the scent portion 30, the scent portion 30 is first hooked on one end of receiving portion 12 of the head 10; then the scent portion 30 is stretched so as to pass over the rear end and the stopper 18 of the head 10 and finally the scent portion 30 is released and its shape is restored as a result of its elasticity such that the scent portion 30 is received and held in the receiving portion 12 of the head 10. The stopper 18 is at least one protrusion to prevent the scent portion 30 from moving backward and off the head 10. FIGS. 5 and 6 show another embodiment that the air freshener has a scent portion 30′ of rectangular shape with a hollow portion 301′ for matching with the receiving portion 12′ of the head 10′. The hollow portion 301′ and the receiving portion 12′ can be of circular, rectangular, elliptic or other suitable shape.

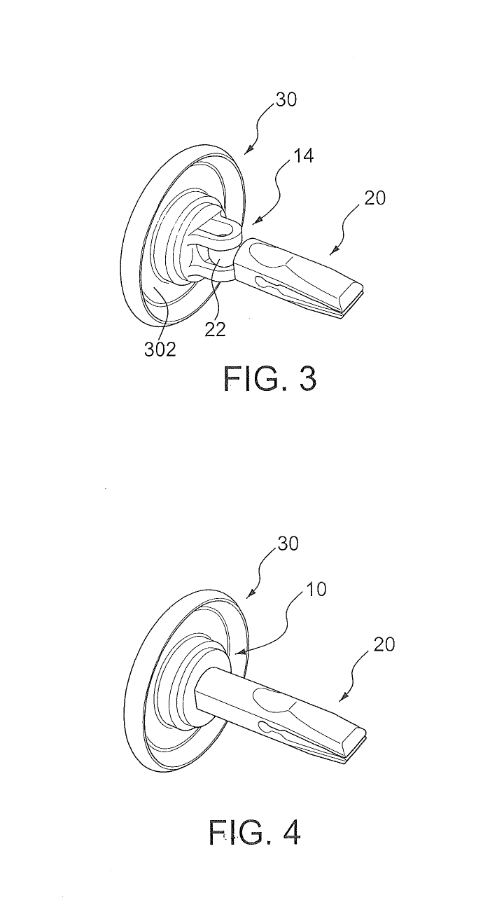

The scent portion 30, 30′ is formed by a scent gel through molding process. The scent gel can be any kind of polymer gel containing scent material, such as polyvinylchloride (PVC), ethylene vinyl acetate (EVA), thermoplastic polyurethane (TPU) and so on. To reduce the shrinkage rate of the polymer during the formation of the scent portion 30 and also to cost down, the scent portion 30 is preferably configured to have a core-out region 302, where the unnecessary polymer material in structure and strength has been removed, as shown in FIGS. 2 and 3.

Another embodiment as shown in FIG. 4 is that the scent portion 30 is fitted to the receiving portion 12 of the head as described above and the clip 20 is extruded from the rear end of the head without any pivot mechanism.

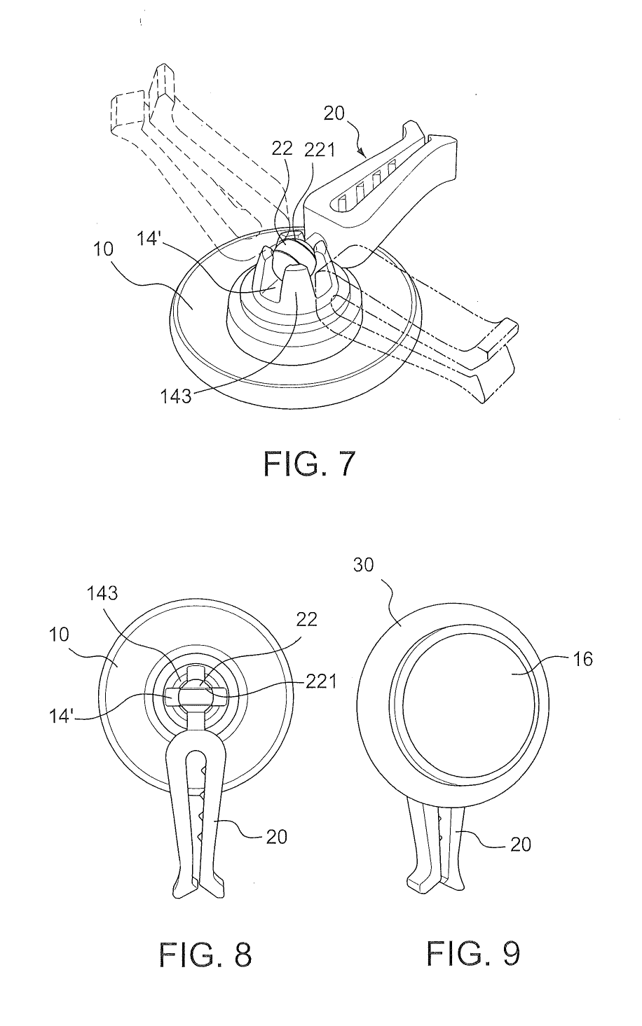

FIGS. 7-9 show an alternative embodiment of the invention. In this embodiment, the socket portion 14′ includes four confining walls 143 respectively extending from the rear end of the head 10 and disposed along the periphery of the socket portion 14′ so as to form a cross-like gap therebetween. The center opening of the cross-like gap is slightly narrower than the diameter of the ball portion 22 and the space formed by the inner surfaces of the four confining walls 143 is able to receive more than half of the ball portion 22, such that the ball portion 22 is pivotably held in the cross-like gap of the socket portion 14′ by means of the four confining walls 143 when the ball portion 22 of the clip 20 is inserted into the cross-like gap of the socket portion 14′. The heights of the four confining walls 143 extending from the rear end of the head are limited and the inner surfaces of the four confining walls 143 are curved so that the ball portion 22 can freely rotate in the space formed by the inner surfaces of the confining walls 143 of the socket portion 14 on the rear side of the head 10; the clip 20 can pivot from at least −90 to +90 degree angle (i.e., more than 180 degree pivot angle) in the cross directions and is pivotable at a smaller angle in other directions.

In an alternative embodiment of the invention, the ball portion 22 of the clip 20 includes at least one rib on its outer surface so as to increase the friction between the confining walls 143 of the socket portion 14′ and the ball portion 22. As a result, when the user inserts the clip 20 into the louver or fin of the air vent and adjusts the head 10 to a desired angle relative to the clip 20, the angle between the head 10 and the clip 20 is maintained and will not occasionally vary due to the weight of the head since the engagement between the ball portion 22 and the socket portion 14′ is enhanced by means of the rib 221.

The front end of the head 10 can further have an exhibition portion 16, as shown in FIGS. 1, 5 and 9. The exhibition portion 16 can be of a rhombus (as shown in FIG. 5) or rectangular shape, or a bulged round surface, a flat round surface (as shown in FIG. 9), a recessed surface (as shown in FIG. 1) or any other desired shape. Patterns or words for decoration or promotion can be directly imprinted or printed on the exhibition portion 16 of the head 10. Patterns or words can also be integrally formed with the head 10 by means of a molding process. In FIGS. 1 and 5, the exhibition portion 16 is a recess and a label or indicia 40 with patterns or words is received therein. The label or indicia 40 can be of any shape fitted to the exhibition portion 16 and adhered to the exhibition portion 16. The label 40 can be further covered with a transparent resin for protection. Another example is that the indicia 40 is made of hard material, such as bronze and aluminum plate, and is relief printed or intaglio printed with the patterns desired. Because the scent portion 30, 30′ is inlaid in the head 10, 10′ and under the indicia or label 40, 40′, the color and shape of the scent portion 30, 30′ can make the indicia or label 40, 40′ much more outstanding and thus have a better exhibitive effect.

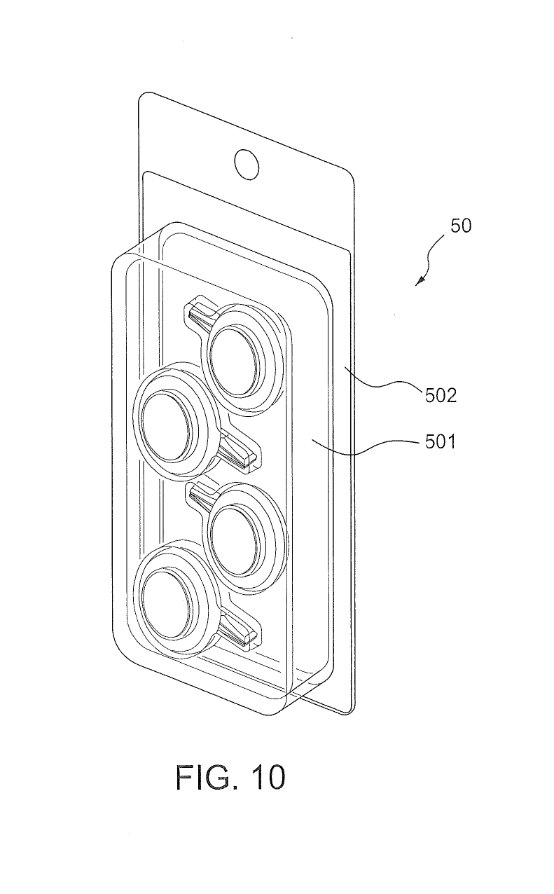

The pivot mechanism adopted in the embodiments shown in FIGS. 1-3, 5-6 and 7-9 benefits a compact package. As shown in FIG. 10, four pieces of the air fresheners as shown in FIG. 3 are received in a package 50 and arranged for display. The package 50 has a transparent shell 501 and a back plate 502. Either the shell 501 or the back plate 502 or both of them has the structure for fixing the air fresheners in proper positions for display. Because the clip 20 of each air freshener has been rotated to be substantially parallel to the top surface of the head 10, the height of the shell 501 is thus reduced.

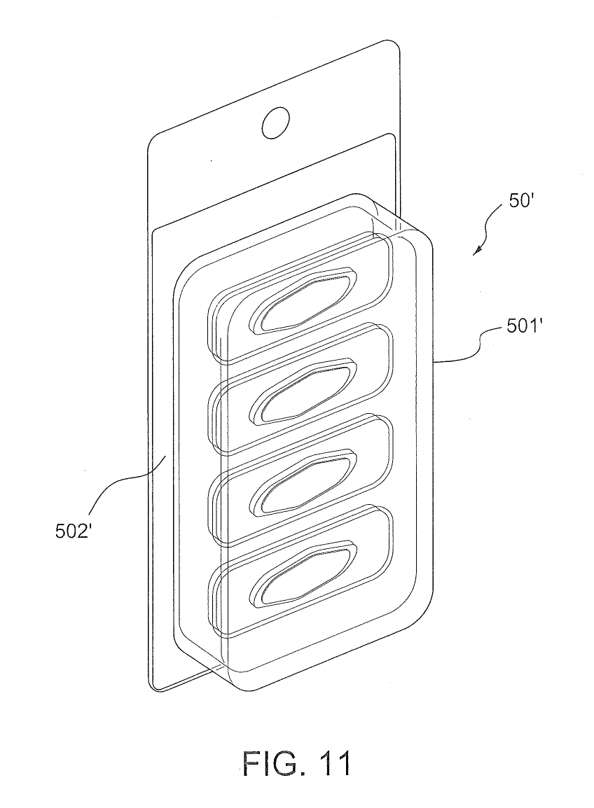

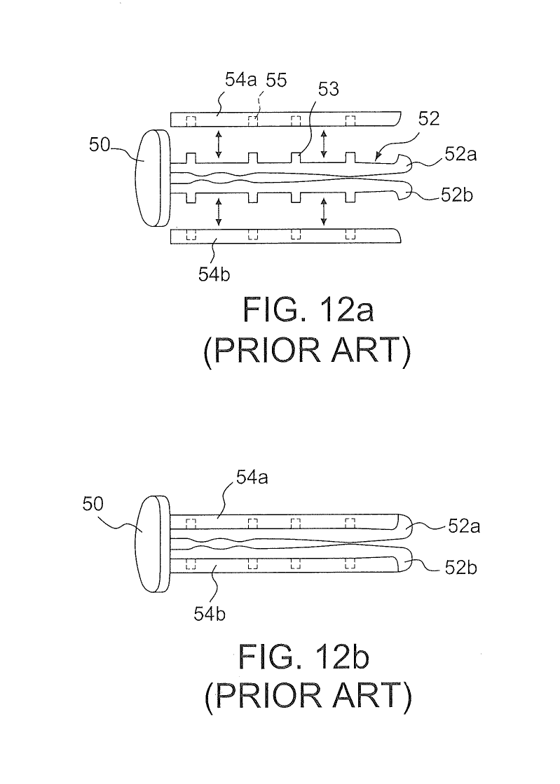

As shown in FIG. 11, four pieces of the air fresheners shown in FIGS. 5 and 6 are arranged in a package 50′ for display. The package 50′ has a transparent shell 501′ and a back plate 502′. Either the shell 501′ or the back plate 502′ or both of them has the structure for fixing the air fresheners in proper positions for display. Because the clip 20′ has been rotated to be substantially parallel to the top surface of the head 10′ and fully hidden from the large scent portion 30′, the height of the shell 501′ is thus reduced. The package 50′ has a compact size and clear-up appearance.

Although the present invention has been described in relation to particular embodiments thereof, many other variations and modifications and other uses may become apparent to those skilled in the art. It is preferred, therefore, that the present invention be limited not by the specific descriptions herein, but only by the appended claims.

What is claimed is:

1. An air freshener, comprising:
a head having a receiving portion recessed from the peripheral surface of the head and a socket portion on its rear end;
a clip having a ball portion on one end, the ball portion being rotatably and detachably received in the socket portion of the head; and
a scent portion at least partially fitted to and received in the receiving portion of the head.

2. An air freshener, comprising:
a head having a receiving portion recessed from the peripheral surface of the head;
a clip extruding from a rear end of the head; and
a scent portion at least partially fitted to and received in the receiving portion of the head.

3. An air freshener according to claim 1 or 2, further comprising a stopper formed between the receiving portion and the rear end of the head.

4. The air freshener according to claim 1, wherein the socket portion has four confining walls respectively extending from the rear end of the head and disposed along the periphery of the socket portion so as to form a cross-like gap therebetween which has a center opening slightly narrower than the diameter of the ball portion and the inner surfaces of the confining walls form a space for receiving more than half of the ball portion such that the clip is pivotably held by the socket portion when the ball portion is inserted in the gap of the socket portion, and the pivot angle of the clip is more than 180 degrees in the cross directions.

5. The air freshener according to claim 4, wherein the heights of the four confining walls extending from the rear end of the head are limited and the inner surfaces of the four confining walls are curved so that the clip can not only pivoted in the cross directions but also slightly pivoted in other directions in a smaller swivel angle.

6. The air freshener according to claim 1, wherein the socket portion has a first wall and a second wall respectively and opposingly extending from the rear end of the head, the first and second walls defining a gap therebetween which is slightly narrower than the diameter of the ball portion, each of the first wall and second wall having a hole, such that the ball portion is pivotably held by the gap and the holes when the ball portion is inserted in the gap of the socket portion.

7. The air freshener according to claim 6, wherein the heights of the first and second walls extending from the rear end of the head are limited and the contours of the first and second walls are curved so that the clip is not only pivotable along the inner surfaces of the first and second walls, but also slightly pivotable in other directions about the socket portion.

8. The air freshener according to claim 4 or 6, wherein the ball portion has at least one rib thereon for increasing the friction between the socket portion and the ball portion.

9. The air freshener according to claim 1 or 2, wherein the scent portion has a hollow portion which is of the shape fit the receiving portion of the head.

10. The air freshener according to claim 1 or 2, wherein the scent portion and the head is formed in any of the following shapes: circle, rectangle, oval, rhombus, square, doll-like shape, heart- shaped and irregular shape.

11. The air freshener according to claim 1 or 2, wherein the scent portion has a core-out region.

12. The air freshener according to claim 1 or 2, wherein the scent portion is at least partially in the form of an elastic band such that the scent portion is at least partially held in the receiving portion by the elasticity of the scent portion.

13. The air freshener according to claim 1 or 2, wherein the head has an exhibition portion on its front end, the exhibition portion having a pattern or words printed, imprinted or integrally formed thereon.

14. The air freshener according to claim 1 or 2, wherein the head has an exhibition portion on its front end, the exhibition portion is configured to be able to receive a label or an indicia.

15. The air freshener according to claim 14, wherein the label is covered with a transparent resin.

16. The air freshener according to claim 14, wherein the label is relief printed.

17. The air freshener according to claim 14, wherein the label is intaglio printed.
